(19) United States
(12) Patent Application Publication
(10) Pub. No.: US 2005/0037348 A1
(43) Pub. Date: Feb. 17, 2005

(54) Charged component detector, its using method and detection panel

(76) Inventors: Akira Tsukada, Shizuoka (JP); Hideharu Mori, Shizuoka (JP); Tatsuya Shinoda, Shizuoka (JP)

Correspondence Address: BIRCH STEWART KOLASCH & BIRCH, PO BOX 747, FALLS CHURCH, VA, 22040-0747, US

(21) Appl. No.: 10/468,227

(22) PCT Filed: Feb. 14, 2002
(86) PCT No.: PCT/JP02/01248

(30) Foreign Application Priority Data
Feb. 19, 2001 (JP) 2001-42032

(51) Int. Cl. C12Q 1/68; C12M 1/34

(52) U.S. Cl. 435/6; 435/287.2; 205/777.5

(57) ABSTRACT

The present invention provides a charged substance detector capable of detecting charged substances easily and quickly without complicating the construction of the detector, a method of using the charged substance detector and a detection panel. The charged substance detector comprises: a detection panel 1 having a plurality of reaction electrodes 2 arrayed in matrix, on which are immobilized reaction charged substances reacting specifically with target charged substances; and a conduction control device 4 having a matrix of wires 3 crossing each other at points corresponding to the matrix-arrayed reaction electrodes 2 of the detection panel 1, and being capable of selectively energizing the reaction electrodes 2 through the matrix of wires 3. Further, to detect a state of reaction in each of the reaction electrodes 2, the conduction control device 4 is provided with a conduction state detector 5. By selectively applying a voltage to a specific reaction electrode 2, the reaction charged substance immobilized on the reaction electrode 2 is allowed to react specifically with a target charged substance. Further, the present invention also covers the detection panel 1 constituting the charged substance detector.

### FIELD OF THE INVENTION

The present invention relates to a charged substance detector for detecting charged substances such as DNA and genes and more particularly to a charged substance detector of a type that utilizes reaction charged substances that specifically react with target charged substances, as well as to a method of using the charged substance detector and a detection panel used in the detector.

### DESCRIPTION OF THE PRIOR ART

Generally, gene sequences of bacteria and viruses are significantly different from those of humans and it is therefore possible to specifically detect bacteria and viruses that are infecting humans by using their specific sequences as criteria.

Such methods for detecting genetic changes include, for example, a DNA probe method.

In this method, a short DNA (DNA probe) having a sequence complementary to a sequence of a gene to be detected is used and labeled with fluorescent materials to determine the presence or absence of the target gene in a sample by its reaction with the gene.

A gene sensor (DNA detector) that quantitatively detects target genes by using the DNA probe method has already been proposed (e.g., in Japanese Patent No. 2573443).

The operation of this gene sensor involves immobilizing a DNA probe that specifically reacts with a target gene to be detected on an electrode, immersing this electrode in a solution containing a sample DNA, forming a DNA hybrid on the electrode by reaction between DNAs, applying an electrochemically active DNA binder (intercalator) to the DNA hybrid formed on the electrode, and detecting the target gene according to an electric signal obtained from the DNA binder.

However, it is assumed that only one gene is detected for each sample because the above mentioned gene sensor, as indicated in Japanese Published Unexamined Patent Application No. 146183/98, has the structure of an independent electrode with a DNA probe immobilized thereon and a reaction with a gene is carried out on that electrode.

If a plurality of genes are detected for each of a plurality of samples, the gene sensors as many as the number at least equal to the number of samples times the number of target genes are needed and it is necessary to perform the gene detection individually using these gene sensors. The gene detection therefore cannot be performed easily and quickly.

A possible method of solving these technical problems may involve providing a plurality of electrodes on a single detection panel and individually wiring the electrodes to enable reactions with genes on the individual electrodes.

In this type, however, since the electrodes are individually wired, different conduction controls equal in number to the electrodes are necessary. With a growing demand for increased number of electrodes, a technical problem like a complicated conduction control arises as consequence.

Such a technical problem is observed not only in the gene detection but also in the common DNA detection and more generally the detection of charged substances such as DNA.

The present invention has been accomplished to solve the above-described technical problems and provides a charged substance detector capable of performing charged substance detection easily and quickly without complicating the construction of the detector, as well as a method of using the same and a detection panel used in the detector.

### DISCLOSURE OF THE INVENTION

As shown in FIG. 1, the present invention provides a charged substance detector comprising: a detection panel 1 having a plurality of reaction electrodes 2 arrayed in matrix, on which are immobilized reaction charged substances reacting specifically with target charged substances; and a conduction control device 4 which has a matrix of wires 3 crossing each other at points corresponding to the matrix-arrayed reaction electrodes 2 of the detection panel 1 and which can selectively energize the reaction electrodes 2 through the matrix of wires 3.

In this technical means, the “charged substances” include not only DNA such as genes but other substances as well.

The detection panel 1 may have reaction electrodes 2 arrayed in matrix and there is no limiting condition on the number of boards. The detection panel 1 may comprise two boards or one board or multiple boards stacked together.

Here, arranging a plurality of reaction electrodes 2 in matrix means that a plurality of reaction electrodes 2 (e.g., four reaction electrodes: D11-D22) are arranged in two vertical columns and two horizontal rows, as shown in FIG. 1.

Further, the conduction control device 4 needs only to selectively energize the reaction electrodes 2 through the matrix of wires 3. The matrix of wires 3 means wires crossing each other at points corresponding to the reaction electrodes 2. For example, column wires X (X1, X2) and row wires Y (Y1, Y2) crossing each other at points corresponding to the reaction electrodes 2 (D11-D22) of 2×2 matrix (2 columns and 2 rows) configuration.

A typical mode for detection panel 1 may, for example, have a two-board construction as described below.

The detection panel 1 comprises a first board having the plurality of reaction electrodes 2 arrayed in matrix, on which are immobilized the reaction charged substances that specifically react with the target charged substances, and the second board opposing the first board and having an electrode to apply a voltage between it and the selected reaction electrode 2 in the matrix.

Further, in this two-board construction, to apply the voltage more flexibly in the detection panel 1, it is preferred that the second board has a plurality of electrodes arranged at positions corresponding to the matrix-arrayed reaction electrodes of the first board.

That is, although the second board may have a single electrode for voltage application, this configuration must select a specific reaction electrode 2 on the first board. On the contrary, if a plurality of separate electrodes are provided on the second board, the flexibility of electrode selection increases, making it possible, for example, to minimize unwanted voltage applications to other reaction electrodes 2 and to select X-column electrodes on the first board and Y-row electrodes on the second board.

Further, while a desired construction may be adopted for the reaction electrodes 2 of the detection panel 1, the reaction electrodes 2, on which are immobilized reaction charged substances that specifically react with target charged substances, typically have a reaction charged substance immobilizing layer. The reaction charged substance immobilizing layer may be provided with an insulating layer as the need arises.

The method of immobilizing the reaction charged substances on the reaction electrodes 2 includes a so-called voltage application method wherein a desired reaction charged substance is immobilized on a specific reaction electrode 2 by applying a voltage to that reaction electrode 2 of the detection panel 1, since each reaction electrode 2 can be selectively energized. It is also possible to use a conventionally used method.

Among the conventionally used methods are an on-chip synthesis method (a method that synthesizes a reaction charged substance, for example DNA, on a board by using photolithography or amidite) and a spotting method (a method that implants a reaction charged substance such as DNA into the board).

Further, the typical mode for conduction control device 4 has switch elements S (S11-S22) connected between the matrix-arrayed reaction electrodes 2 and the matrix of wires 3 and selectively turns on or off the switch elements S to apply a predetermined voltage to a specific reaction electrode 2.

To detect a state of reaction in the reaction electrode 2, the conduction control device 4 is provided with a conduction state detector 5 that detects a conduction state of the reaction electrode 2.

Next, a method of using the charged substance detector of the present invention will be explained.

To determine whether the same charged substance is present in a plurality of samples, a typical method of using the charged substance detector includes immobilizing a predetermined reaction charged substance on a required number of reaction electrodes 2 of the detection panel 1 and successively applying a predetermined voltage to the specific reaction electrodes 2 corresponding to each of the samples to successively allow the predetermined reaction charged substance to react specifically with the target charged substance in each sample.

Also, to determine whether a plurality of charged substances are present in one sample, a typical method of using the charged substance detector includes successively immobilizing different reaction charged substances on each of the reaction electrodes 2 of the detection panel 1 and applying a predetermined voltage to each of the reaction electrodes 2 to successively allow each of the reaction charged substances to react specifically with the corresponding target charged substances in the sample.

Further, to determine whether a plurality of charged substances are present in a plurality of samples, a typical method of using the charged substance detector includes successively immobilizing different reaction charged substances on different reaction electrodes 2 of the detection panel 1 and applying a predetermined voltage to each of the reaction electrodes 2 corresponding to each of the samples to successively allow the reaction charged substances on the reaction electrodes 2 to react specifically with the corresponding target charged substances in the samples.

In addition to the charged substance detector and the method of using the same, the present invention also covers the detection panel 1 constituting the charged substance detector.

In this case, the present invention relates to detection panel 1 wherein the reaction charged substances which correspond to the number of inspection items are assigned and immobilized on predetermined addresses of the reaction electrodes.

With this mode, in an inspection that has predetermined inspection items, it is possible to template the detection panel 1 into a fixed form.

### BRIEF DESCRIPTION OF DRAWINGS

FIG. 1 is an explanatory diagram showing an outline construction of a charged substance detector according to the present invention.

FIG. 2(a) is an explanatory diagram showing an overall construction of a DNA detector according to Embodiment 1; FIG. 2(b) is an exploded perspective view showing an outline construction of a detection panel; and FIG. 2(c) is an explanatory diagram showing an example of a method for filling a liquid into a hollow portion of the detection panel.

FIG. 3(a) is an explanatory diagram showing an example construction of an upper board of the detection panel used in the DNA detector according to Embodiment 1; and FIG. 3(b) is an explanatory diagram showing an example construction of a lower board of the detection panel.

FIG. 4 is a schematic cross-sectional view of the detection panel used in Embodiment 1.

FIG. 5 is an example construction of a conduction control circuit for the detection panel used in Embodiment 1.

FIG. 6 is an explanatory diagram showing how DNA probes are immobilized on the detection panel of the DNA detector used in Embodiment 1.

FIG. 7 is an explanatory diagram showing an example use of the DNA detector as used in Embodiment 1.

FIG. 8 is an explanatory diagram showing an example output of a current detector used in Embodiment 1.

FIG. 9(a) is an explanatory diagram showing an example construction of an upper board of the detection panel used in the DNA detector according to Embodiment 2.

FIG. 9(b) is an explanatory diagram showing an example construction of a lower board of the detection panel.

FIG. 10 is a cross-sectional view of the detection panel used in Embodiment 2.

FIG. 11 is an explanatory diagram showing an example construction of a conduction control circuit for the detection panel used in Embodiment 2.

FIG. 12(a) is an explanatory diagram showing an outline construction of a DNA detector according to Embodiment 3.

FIG. 12(b) is an exploded perspective view showing an outline construction of a detection panel.

FIG. 12(c) is an explanatory diagram showing an example of a method for filling a liquid into a hollow portion of the detection panel.

FIG. 13 is an explanatory diagram showing an outline construction of a DNA detector according to Embodiment 4.

FIG. 14(a) is an explanatory diagram showing an outline construction of a DNA detector according to Embodiment 5.

FIG. 14(b) is a view as seen from a direction B in FIG. 14(a).

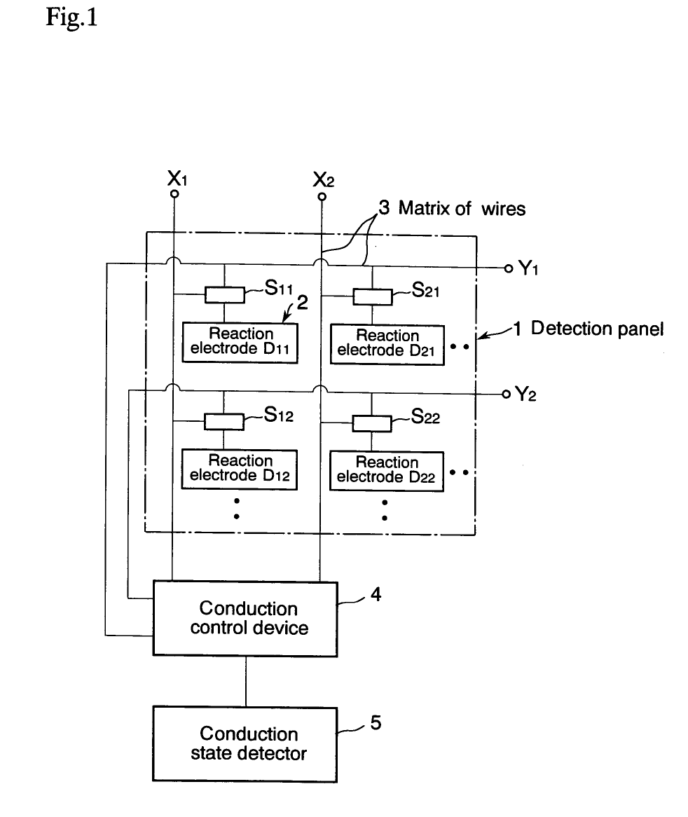

### BEST MODE FOR CARRYING OUT THE INVENTION

Embodiment 1

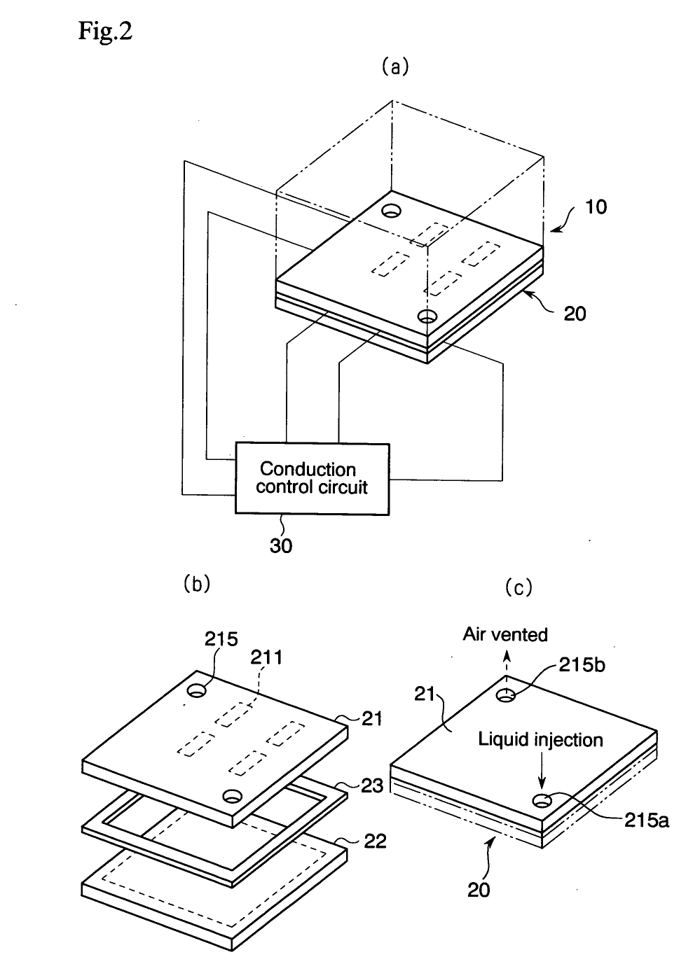

FIG. 2A is an explanatory diagram showing an outline of Embodiment 1 of a DNA detector according to the present invention.

In the figure, the DNA detector has a boxed-shaped well 10 (indicated by an imaginary line in FIG. 2(a)) with openings in its upper surface for receiving a sample and a reaction solution. At the bottom of the well 10 are provided a detection panel 20 for detecting a DNA and a conduction control circuit 30 for controlling the energization of the detection panel 20.

In this embodiment, the detection panel 20, as shown in FIG. 2(b), has an upper board 21 and a lower board 22 arranged apart from each other by a spacer 23 installed there to form a hollow portion between the boards.

In this embodiment, the upper board 21 has a plurality (in this case two) of communication holes 215 formed therein at diagonal positions, through which a liquid (such as a sample or specimen) to be loaded into the well 10 is filled into the hollow portion between the upper and lower boards 21 and 22 of the detection panel 20.

More specifically, as shown in FIG. 2(c), the liquid is poured in through one communication hole 215a, with the other communication hole 215b functioning as an air vent. In this case, the hollow portion in the detection panel 20 is filled with the liquid through a capillary action.

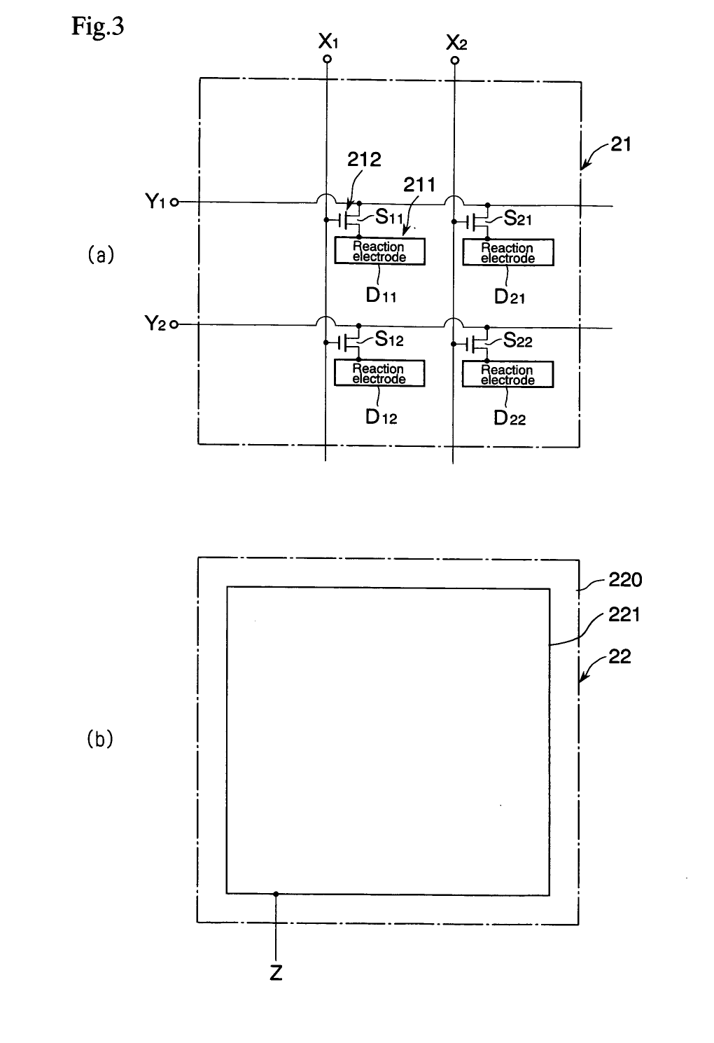

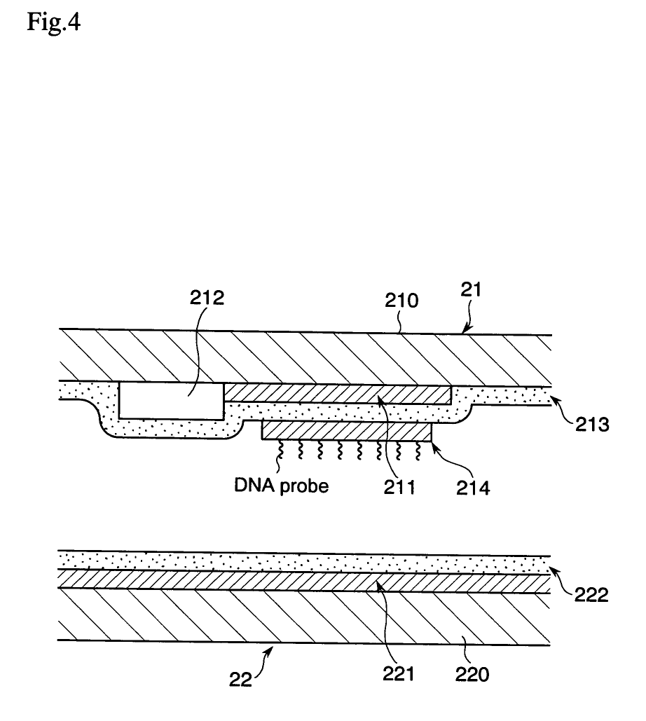

As shown in FIG. 3(a) and FIG. 4 in particular, the upper board 21 has a plurality of reaction electrodes 211 (D11-D22) arranged in, for example, a 2×2 matrix on an insulating rectangular base substrate 210. Column wires X (X1, X2) extending in a column direction (or vertical direction) corresponding to the reaction electrode 211 (D11-D22) and row wires Y (Y1, Y2) extending in a row direction (or horizontal direction) corresponding to the reaction electrode 211 (D11-D22) to cross the column wires X are arranged. Switch elements 212 (S11-S22) are connected between each of the reaction electrodes 211 (D11-D22) and the wires X, Y.

In this embodiment, TFTs (thin film transistors) are used as the switch elements 212. The column wires X (X1, X2) corresponding to the reaction electrodes 211 (D11-D22) are connected to gates of the TFTs, the row wires Y (Y1, Y2) are connected to sources of the TFTs, and the reaction electrodes 211 (D11-D22) are connected to drains of the TFTs.

The base substrate 210 of the upper board 21 is covered with an insulating film 213 over an entire area of the reaction electrodes 211 and the wires (X, Y), and DNA immobilizing layers 214 are placed on those areas of the insulating film 213 that correspond to the reaction electrodes 211.

In this embodiment, a polyvinyl acetate film or other resin films are used as the DNA immobilizing layers 214.

While this embodiment has the entire area of the reaction electrodes 211 and the wires (X, Y) covered with the insulating film 213, the insulating film 213 is provided according to the use of the target area. For example, the DNA immobilizing layers 214 may be mounted directly on the reaction electrodes 211.

As shown in FIG. 3(b) and FIG. 4 in particular, the lower board 22 has an insulating rectangular base substrate 220 on which a rectangular counter electrode 221 slightly smaller than the base substrate 220 is mounted. The counter electrode 221 is connected to a power wire Z. An entire area of the counter electrode 221 on the base substrate 220 may be covered with an insulating film 222 according to the use.

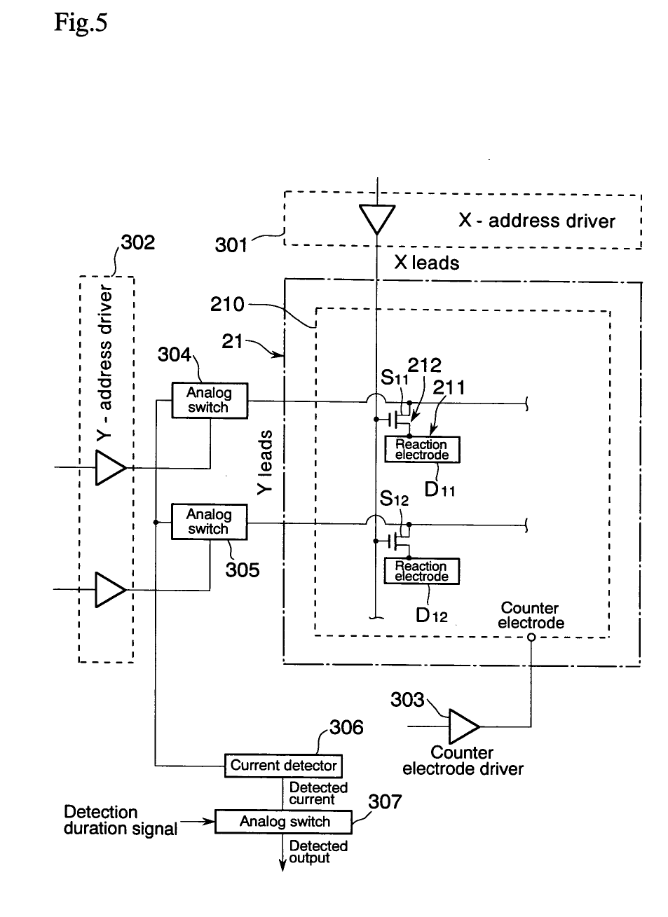

Further, the conduction control circuit 30 for the detection panel 20 may be constructed as shown in FIG. 5.

In the figure, the column wires X (X leads) are turned on or off by a drive signal from an X-address driver 301 and the row wires Y (Y leads) are turned on or off by a drive signal from a Y-address driver 302. Further, the counter electrode 221 is turned on or off by a drive signal from a counter electrode driver 303.

Also, the row wires Y (Y leads) are provided with analog switches 304 and 305 that are turned on or off by the drive signal from the Y-address driver 302. A current detector 306 is connected in series with each of the analog switches 304 and 305.

In this embodiment, in particular, the current detector 306 is connected with an analog switch 307 that is turned on according to a detection duration signal to output an appropriate part of the detected current from the current detector 306 as a detection output.

Next, a method of immobilizing a DNA probe on the detection panel of the DNA detector according to the present embodiment will be described.

In this embodiment, since the detection panel 20 has four reaction electrodes 211 (D11-D22) that can be selectively energized, it is possible to immobilize different DNA probes on individual reaction electrodes 211 (D11-D22) by selectively applying a voltage to each reaction electrode 211.

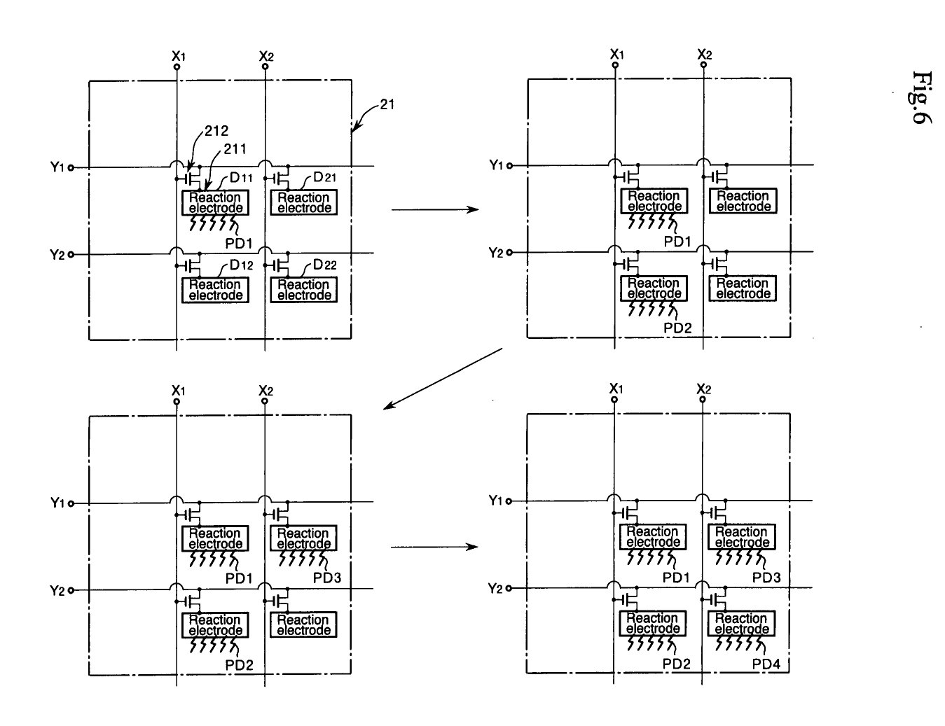

Now, the method of immobilizing different DNA probes on four reaction electrodes 211 (D11-D22) of the detection panel 20 will be described. As shown in FIG. 6, first, wires (X1, Y1) of the corresponding address are activated to apply a positive voltage to the reaction electrode 211 (D11). In this state, a sample containing a first DNA probe PD1 is poured into the hollow portion of the detection panel 20 where the first DNA probe PD1 is bound covalently to only the reaction electrode 211 (D11) by an electric field present between the reaction electrode 211 (D11) and the counter electrode 221.

Next, after the sample in the hollow portion of the detection panel 20 is removed and washed away, wires (X1, Y2) are activated to apply a positive voltage to the reaction electrode 211 (D12). In this state, a sample containing a second DNA probe PD2 is filled into the hollow portion of the detection panel 20 where the second DNA probe PD2 is bound covalently to only the reaction electrode 211 (D12) by an electric field present between the reaction electrode 211 (D12) and the counter electrode 221.

Further, after the sample in the hollow portion of the detection panel 20 is removed and washed away, wires (X2, Y1) of the corresponding address are activated to apply a positive voltage to the reaction electrode 211 (D21). In this state, a sample containing a third DNA probe PD3 is filled into the hollow portion of the detection panel 20 where the third DNA probe PD3 is bound covalently to only the reaction electrode 211 (D21) by an electric field present between the reaction electrode 211 (D21) and the counter electrode 221.

As a last step, after the sample in the hollow portion of the detection panel 20 is removed and washed away, wires (X2, Y2) of the corresponding address are activated to apply a positive voltage to the reaction electrode 211 (D22). In this state, a sample containing a fourth DNA probe PD4 is filled into the hollow portion of the detection panel 20 where the fourth DNA probe PD4 is bound covalently to only the reaction electrode 211 (D22) by an electric field present between the reaction electrode 211 (D22) and the counter electrode 221. After this, the sample filled in the hollow portion of the detection panel 20 is removed and washed away.

The DNA probes PD1-PD4 may be selected from HIV (human immunodeficiency virus), HCV (hepatitis C virus), HBs-Ab (hepatitis B surface-antibody) and HBs-Ag (hepatitis B surface-antigen), according to the purpose of the inspection.

Also to securely immobilize the DNA probes PD (PD1-PD4) on the corresponding reaction electrodes 211 (D11-D22), it is preferred that a negative voltage be positively applied to all other reaction electrodes 211 than the one on which the DNA probe PD of interest is to be immobilized, so that the DNA probe PD of interest is electrically repelled from other reaction electrodes 211.

The above-mentioned performance of the DNA detector of this embodiment is verified by Example 1 described later.

In this embodiment, of course, conventional methods for immobilizing the DNA probes on the detection panel 20 (e.g., an on-chip synthesis method and a spotting method) may be used.

Next, the use of the DNA detector according to the present embodiment will be described.

By using the DNA detector in which four different DNA probes PD (PD1-PD4) are immobilized on the four reaction electrodes 211 (D11-D22), a process of determining whether a target DNA is present or not in a sample DNA will be explained.

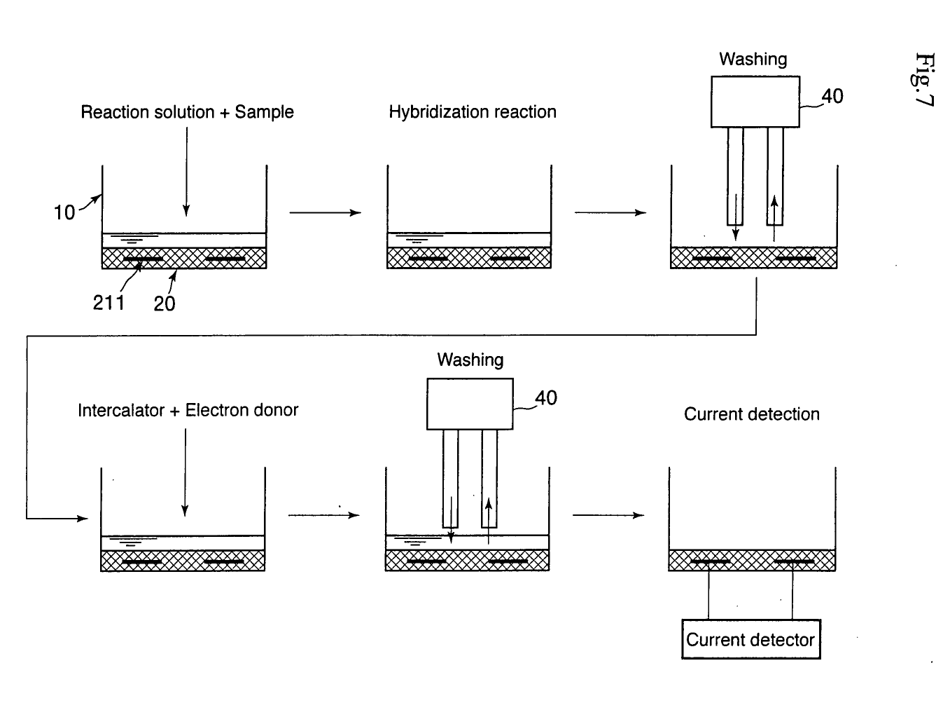

In FIG. 7, first, the wires (X1, Y1), (X1, Y2), (X2, Y1), (X2, Y2) of the addresses corresponding to all the reaction electrodes 211 (D11-D22) of the detection panel 20 are made active to apply a positive voltage to all the reaction electrodes 211 (D11-D22). In this state, a sample and a reaction solution (a solution to promote the reaction of hybridization) are poured into the well 10 to fill the hollow portion of the detection panel 20.

Then, the DNA probes are allowed to hybridize with target DNAs in the sample for a predetermined period of time at a specified temperature.

Then, a washing device 40 is used to remove and washed away the sample and reaction solution from the well 10, and a solution of an intercalator and an electron donor is poured into the well 10 to fill the hollow portion of the detection panel 20.

At this time, in the reaction electrodes 211 (D11-D22) on which the hybridization reaction has occurred, electrons are led to a drain electrode.

Then, the solution in the well 10 is removed again and washed away by the washing device 40. This is followed by successively activating the wires (X1, Y1), (X1, Y2), (X2, Y1), (X2, Y2) of the addresses corresponding to the reaction electrodes 211 (D11-D22) to detect by the current detector 306 a current produced in each of the reaction electrodes 211 (D11-D22)corresponding to a degree of the hybridization reaction.

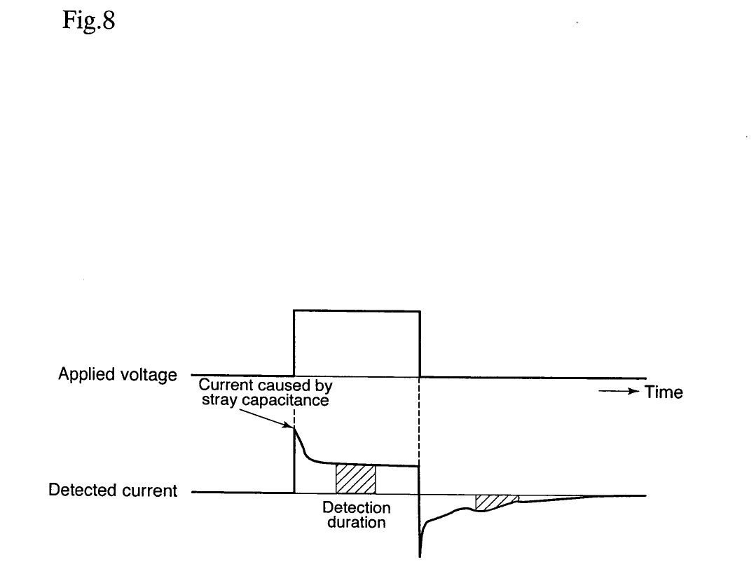

Here, since the current detected by the current detector 306 includes a current not involved in the target reaction such as a current produced by wire capacitances and unwanted ions in the solution, as shown in FIG. 8, this embodiment picks up only a current change involved in the hybridization reaction by excluding unwanted detection periods by the analog switch 307 that is turned on by a detection duration signal.

A current change in each of the reaction electrodes 211 (D11-D22) can be detected by turning on and off a voltage applied to the individual reaction electrodes 211 (D11-D22). Therefore, it becomes possible to determine on which of the reaction electrodes 211 (D11-D22) the hybridization reaction has occurred and to identify a target DNA in the sample.

Embodiment 2

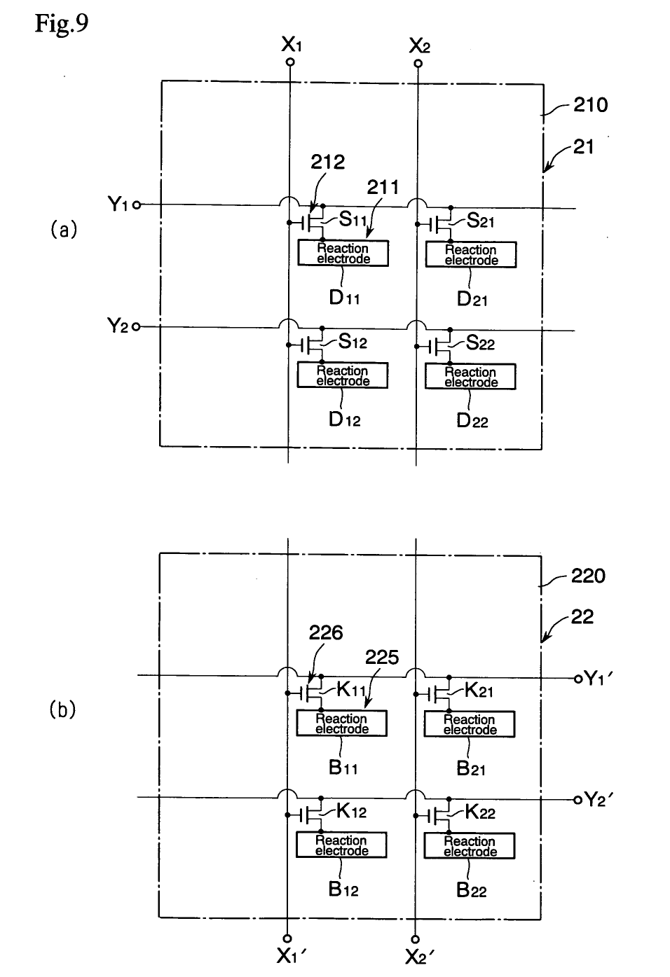

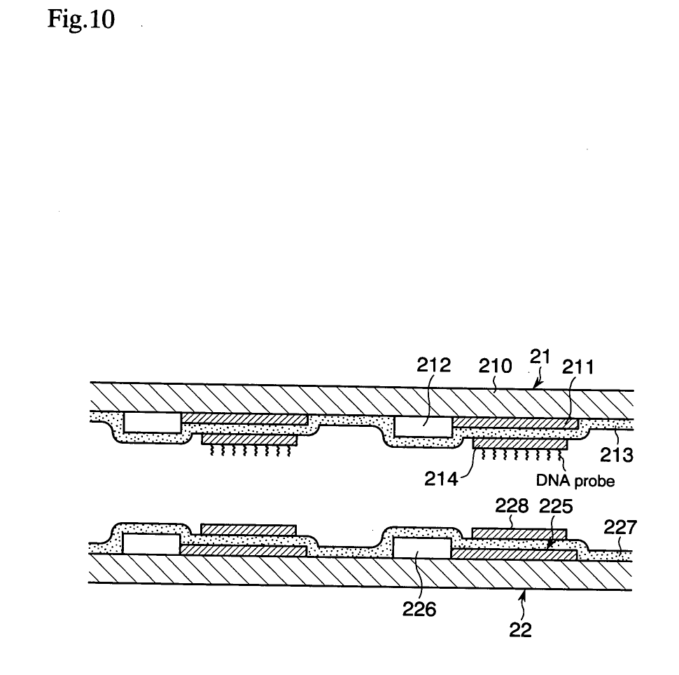

FIGS. 9 to 11 are explanatory diagrams showing a detection panel of the DNA detector and a conduction control circuit for the detection panel according to Embodiment 2.

The basic construction of the detection panel 20 of this embodiment is almost similar to that of Embodiment 1, in that an upper board 21 and a lower board 22 are stacked together with a spacer 23 interposed there to form a hollow portion in the panel. The only difference from Embodiment 1 is the construction of the lower board 22. In this embodiment, constitutional elements similar to those of Embodiment 1 are given with the same numerals as Embodiment 1 and their detailed descriptions are omitted here.

In this embodiment, in the case that the detection panel 20 has the same upper board 21 as that of Embodiment 1 (which, for example, has a 2×2 matrix of reaction electrodes 211 (e.g., D11-D22)). Then the lower board 22 of this embodiment has a plurality of counter electrodes 225 (e.g., B11-B22) arranged on an insulating rectangular base substrate 220 at the opposing areas to the reaction electrodes 211 (D11-D22) of the upper board 21. Column wires X′ (X1′, X2′) extending in a column direction (or vertical direction) corresponding to the counter electrodes 225 (B11-B22) and row wires Y′ (Y1′, Y2′) extending in a row direction (or horizontal direction) corresponding to the counter electrodes 225 to cross the column wires X′ are arranged. Switch elements 226 (K11-K22) are connected between each of the counter electrodes 225 (B11-B22) and the wires X′, Y′.

In this embodiment, TFTs (thin film transistors) are used as the switch elements 226. The column wires X′ (X1′, X2′) corresponding to the counter electrodes 225 (B11-B22) are connected to gates of the TFTs, respectively, the row wires Y′ (Y1′, Y2′) corresponding to the counter electrodes 225 are connected to sources of the TFTs, respectively, and the counter electrodes 225 (B11-B22) are connected to drains of the TFTs.

Then, the lower board 22 is covered with an insulating film 227 over a part or an entire area of the counter electrodes 225 and the wires (X′, Y′) on the base substrate 220, as the need arises.

In this embodiment, as the lower board 22, the upper board 21 is used without change, therefore, DNA immobilizing layers 228 similar to those used on the upper board 21 are provided at areas corresponding to the counter electrodes 225.

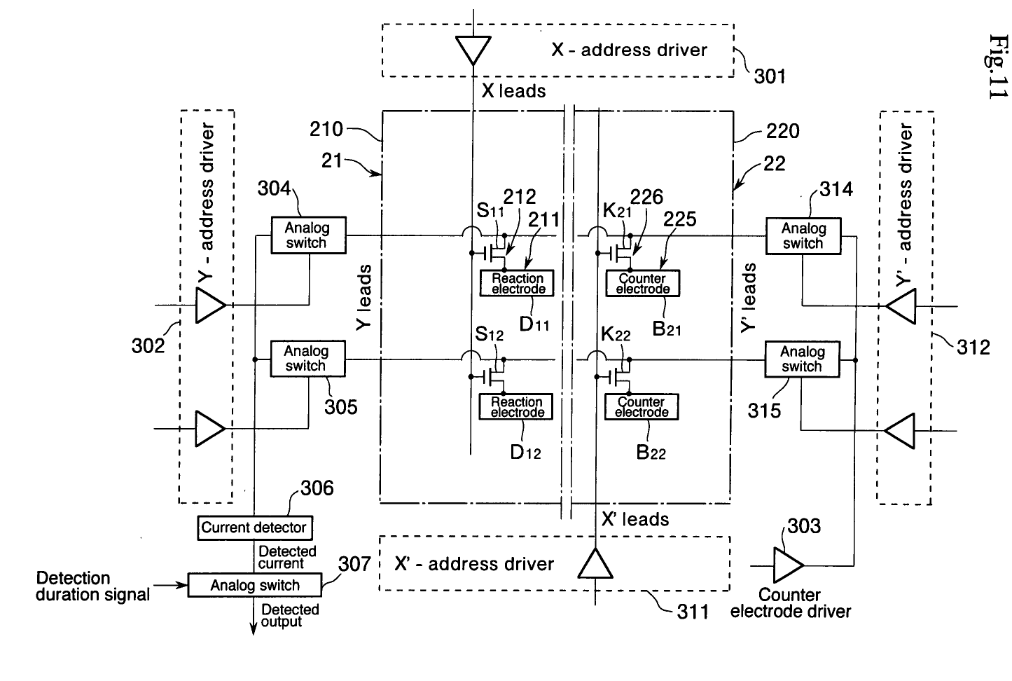

Further, the conduction control circuit 30 for the detection panel 20 is constructed as shown in FIG. 11. In this embodiment, constitutional elements similar to those of Embodiment 1 are given with the same numerals and their detailed explanations are omitted here.

In the figure, while the conduction control circuit 30 for the upper board 21 is the same as that of Embodiment 1, the conduction control circuit 30 for the lower board 22 is different from that of Embodiment 1. That is, the column wires X′ (X′ leads) are turned on or off by a drive signal from an X′-address driver 311 and the row wires Y′ (Y′ leads) are turned on or off by a drive signal from a Y′-address driver 312. The row wires Y′ (Y′ leads) are also provided with analog switches 314 and 315 that are turned on or off by the drive signal from the Y′-address driver 312. A counter electrode driver 303 is connected in series with each of the analog switches 314 and 315.

With this embodiment, since the lower board 22 of the detection panel 20 is provided with counter electrodes 225 (K11-K22) corresponding to the reaction electrodes 211 (D11-D22) and since the counter electrodes 225 (K11-K22) can be energized individually, the detection panel 20 can be operated in the same way as in Embodiment 1 by individually selecting the reaction electrodes 211 (D11-D22) on the upper board 21 and energizing all the counter electrodes 225 on the lower board 22. Other operational modes are also possible. Individually selecting the counter electrodes 225 as well as the reaction electrodes 211 can greatly reduce an accident of a voltage being undesirably applied between the reaction electrodes 211 and the counter electrodes 225, thus selection of the reaction electrodes 211 is carried out correctly.

Embodiment 3

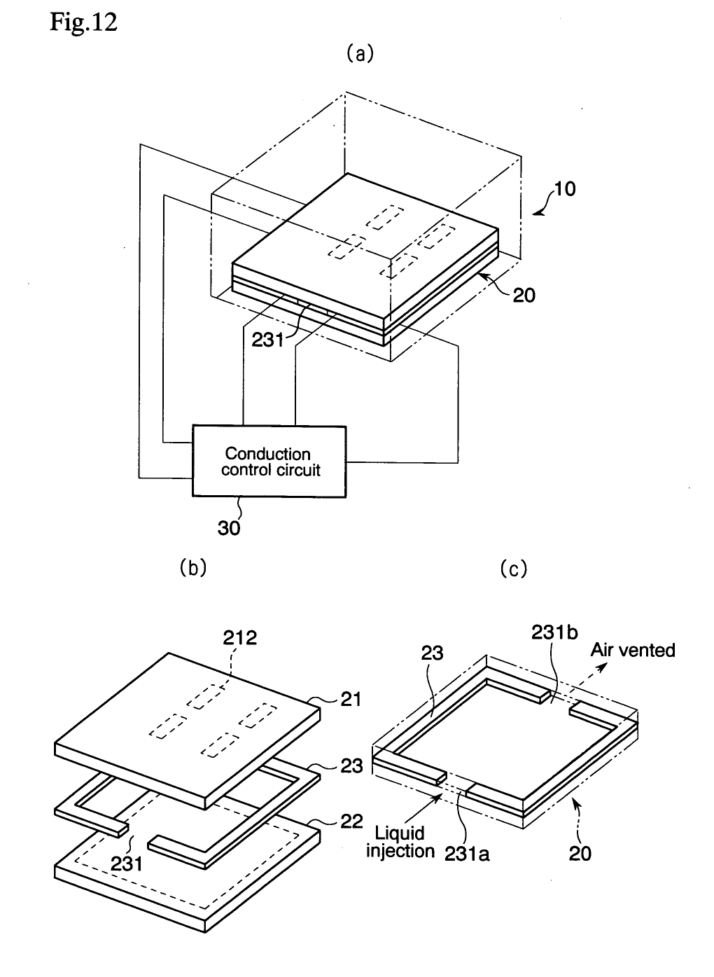

FIG. 12(a) illustrates Embodiment 3 of a DNA detector according to the present invention.

In the figure, the basic construction of the DNA detector is similar to that of Embodiment 1, in that the detection panel 20 is arranged at the bottom portion of the well 10. However, this embodiment differs from Embodiment 1 in the construction of the detection panel 20.

In this embodiment, the detection panel 20, as shown in FIG. 12(a) and 12(b), is a rectangular plate slightly smaller than a bottom inner area of the well 10 and has an upper board 21 and a lower board 22 arranged part from each other by a spacer 23 installed there to form a hollow portion between the boards. Rather than forming the communication holes 215 in the upper board 21 as in Embodiment 1 (see FIG. 2), the spacer 23 is formed with a plurality of notched openings 231 (231a, 231b in FIG. 12(c)) at, for example, two opposite locations thereof.

Therefore, according to this embodiment, as shown in FIG. 12(c), a liquid such as a sample is filled into the hollow portion of the detection panel 20, for example, by injecting the liquid from one notched opening 231a of the spacer 23, with the other notched opening 231b of the spacer 23 used as an air vent in the well 10. In this case, the hollow portion in the detection panel 20 is filled with the liquid through a capillary action.

Embodiment 4

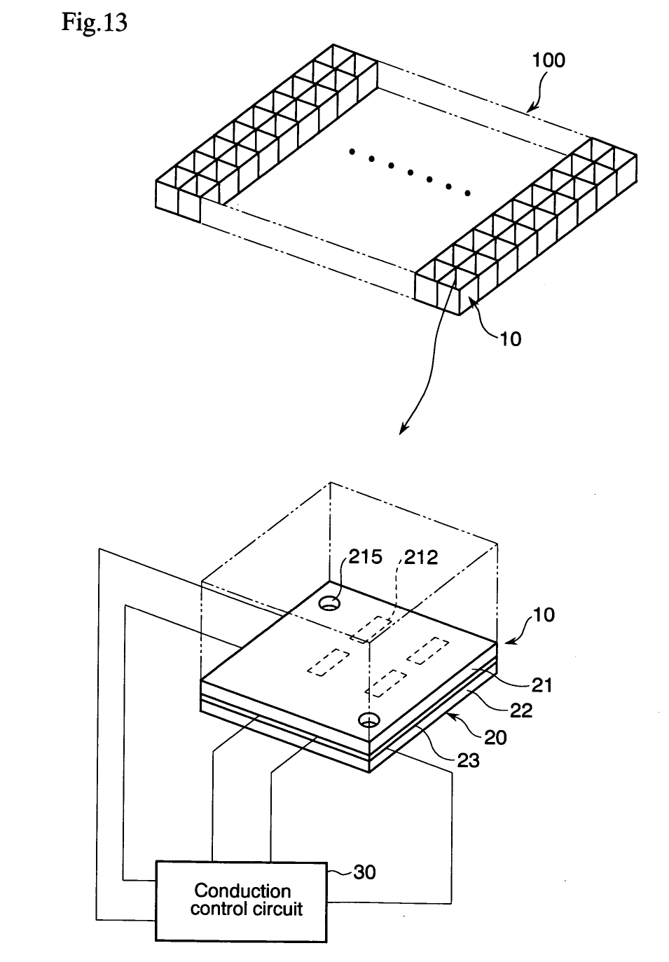

FIG. 13 illustrates Embodiment 4 of a DNA detector according to the present invention.

A DNA detector according to this embodiment represents an effective mode for detecting a plurality of target DNA for a plurality of samples.

In the figure, the DNA detector is provided in the form of a multiplate 100 having a plurality of wells 10 joined together, for example, in an 8×12 matrix. Each of the wells 10 has a detection panel 20, such as shown in Embodiment 1, installed at the bottom thereof and connected with a conduction control circuit 30.

With this embodiment, a plurality of samples are poured into individual wells 10 of the multiplate 100 so that the presence or absence of a plurality of target DNA can be checked for each sample by the detection panel 20 in each well 10.

Embodiment 5

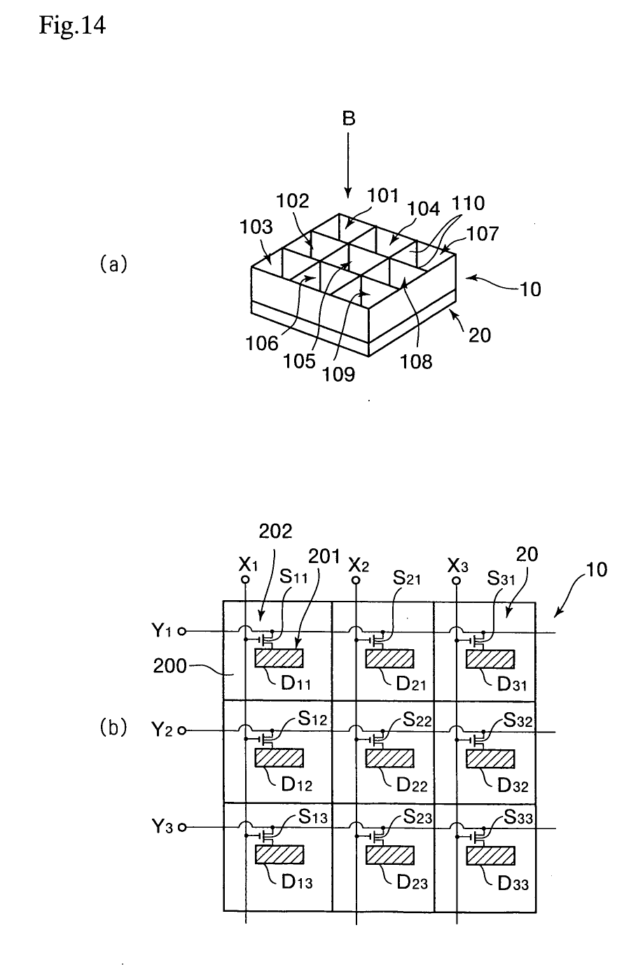

FIGS. 14(a) and 14(b) illustrate Embodiment 5 of a DNA detector according to the present invention.

A DNA detector according to this embodiment represents an effective mode for detecting the same target DNA for a plurality of samples.

In the figure, the DNA detector has one well 10 divided by partitions 110 into a plurality of sub-wells, for example nine sub-wells, in each of which a detection panel 20 is installed on a well bottom.

In this embodiment, the detection panel 20, as shown in FIG. 14(b), has a plurality of reaction electrodes 201 (D11-D33) arranged, for example, in a 3×3 matrix on a base substrate 200. On the base substrate 200, column wires X (X1, X2, X3) extending in a column direction corresponding to the reaction electrodes 201 and row wires Y (Y1, Y2, Y3) extending in a row direction to cross the column wires X are arranged. Switch elements 202 (S11-S33) consisting of TFTs are connected between each of the reaction electrodes 201 and the wires (X, Y) to connect the reaction electrodes to a conduction control circuit not shown in the figure. The reaction electrodes 201 and the wires (X, Y) are covered with an insulating film not shown in the figure. DNA immobilizing layers not shown in the figure are placed on the insulating film at those sites that correspond to the reaction electrodes 211 and DNA probes are immobilized on the layers.

In this embodiment in particular, the reaction electrodes 201 (D11-D33) of the detection panel 20 are arranged at positions corresponding to sub-wells 101-109 separated from each other by partitions 110 of the well 10. At the bottom of each sub-well 101-109 a DNA immobilizing layer with DNA probes immobilized thereon is exposed.

With the DNA detector according to this embodiment, different samples are poured into the sub-wells 101-109 of the well 10 for hybridization reactions. Then, a voltage is successively applied to the reaction electrodes 201 (D11-D33) to detect a current from each reaction electrode 201. This makes it possible to evaluate in which of the sub-wells 101-109 the sample has hybridized with the DNA probe, thereby determining the presence or absence of a target DNA.

### EXAMPLE 1

Using the DNA detector according to Embodiment 1, an experiment was conducted to immobilize DNA probes on the reaction electrodes 211 of the upper board 21 (in this example, four reaction electrodes D11-D22 in 2×2 matrix are provided). It was found that desired DNA probes were immobilized on arbitrary reaction electrodes 211 (D11-D22), as described below.

In this example, the following DNA probes were synthesized:

(SEQ ID NO: 1)

Sequence of DNA probe a: 5′-GACGGAACAGCTTTGAGGTGC;
and

(SEQ ID NO: 2)

Sequence of DNA probe b: 5′-TGACGGAGGTTGTGAGGC.

DNA probes a and b have an amino group at 5′ terminal with a spacer there. The DNA immobilizing layer of the upper board 21 is a film made of polyvinyl acetate resin and has a carboxyl group on its surface.

[Information on Artificial Sequence <223>]: Synthesized DNA

In this example, addressed wires (X1, Y1) were selected to apply a positive voltage of between 0.5 V and 2 V to only a reaction electrode 211 (D11). Other reaction electrodes 211 were applied a negative voltage. The hollow portion (the hollow portion between the upper board 21 and the lower board 22) of the detection panel 20 was filled with a boronic acid buffer containing 0.1 mmol/L of DNA probe a and 5 mmol/L of water-soluble carbadiimide (50 mmol/L, pH8.0). In this state, the detection panel 20 was heated to 37° C. and allowed to stand for 10 minutes. This reaction resulted in the DNA probe a bound covalently to only the reaction electrode D11.

After this, all the reaction electrodes 211 including the reaction electrode D11 were set to a negative potential and the hollow portion of the detection panel 20 was washed using a boronic acid buffer (50 mmol/L, pH8.0) to remove the DNA probe a that failed to bind covalently to the reaction electrode D11.

Next, addressed wires (X2, Y1) were selected to apply a positive voltage of between 0.5 and 2 V to only the reaction electrode D12. Other reaction electrodes were applied a negative voltage. The hollow portion of the detection panel 20 was filled with a boronic acid buffer containing 0.1 mmol/L of DNA probe b and 5 mmol/L of water-soluble carbadiimide. In this state, the detection panel 20 was heated to 37° C. and allowed to stand for 10 minutes. This reaction resulted in the DNA probe b binding covalently to only the reaction electrode D12.

Then, all the reaction electrodes 211 were set to a negative potential and the hollow portion of the detection panel 20 was washed with a boronic acid buffer to remove the DNA probe b that failed to bind covalently to the reaction electrode D12.

In this example, although DNA probes were not bound to the reaction electrodes D21 and D22, a desired DNA probe may be bound to any reaction electrode 211 by repeating the above-described process.

### EXPERIMENT 2

Using the DNA detector prepared in Example 1, another experiment was conducted to check for the presence or absence of hybridization reactions with a plurality of sample DNA.

(SEQ ID NO: 3)

Sequence of sample DNA1: 5′-GCACCTCAAAGCTGTTCCGTC

(SEQ ID NO: 4)

Sequence of sample DNA2: 5′-GCCTCACAACCTCCGTCA

(SEQ ID NO: 5)

Sequence of sample DNA3: 5′-GCACAGAGGAAGAGAATCTCC

The sample DNA1 is complementary to the DNA probe a and the sample DNA2 is complementary to the DNA probe b. There is no probe on the detection panel 20 that is complementary to the sample DNA3.

Then, the following four kinds of liquid mixture of sample DNA were prepared.

Liquid mixture 1: Tris-hydrochloric acid buffer (10 mmol/L, pH8.0) containing 1 μmol/L of sample DNA1;

Liquid mixture 2: Tris-hydrochloric acid buffer (10 mmol/L, pH8.0) containing 1 μmol/L of sample DNA2;

Liquid mixture 3: Tris-hydrochloric acid buffer (10 mmol/L, pH8.0) containing 1 μmol/L each of sample DNA1 and sample DNA2; and

Liquid mixture 4: Tris-hydrochloric acid buffer (10 mmol/L, pH8.0) containing 1 μmol/L of sample DNA3.

[Information on Artificial Sequence <223>]: Synthesized DNA

First, all the reaction electrodes 211 (D11-D22) of the detection panel 20 were applied a positive voltage and the hollow portion of the detection panel 20 (the hollow portion between the upper board 21 and the lower board 22) was filled with the liquid mixture 1. It was kept for Hybridization reaction was carried out at 50° C. for 10 minutes.

After the reaction, all the reaction electrodes 211 (D11-D22) were applied a negative voltage and the hollow portion was washed with a tris-hydrochloric acid buffer (10 mmol/L, pH8.0).

Next, 0.1 mol/L of Hoechst 33258 (Molecular Probes, Inc.) solution was poured into the detection panel and allow to stand in a dark place for five minutes.

After the detection panel was washed with a tris-hydrochloric acid buffer (10 mmol/L, pH8.0), a current signal was detected by the current detector 306.

There are two detection methods available: one is to use only the reaction electrodes 211 on the upper board 21 and measure an oxidation current produced by Hoechst 33258 (method 1); and the other involves applying a voltage between the reaction electrodes 211 and the counter electrode 221 and measuring a current that flows (method 2).

First, the method 1 was used and addressed wires (X1, Y1) were selected to measure a current value of the reaction electrode D11. An appropriate portion of an observed current waveform was detected by manipulating the analog switch 307. The current values for the reaction electrodes D12 and D21 were similarly measured.

Using the method 2, a voltage was applied between each of the reaction electrodes 211 (D11-D21) and the counter electrode 221 to measure current values of the reaction electrodes D11-D21.

Then, in a manner described above, the current values in the reaction electrodes D11, D12 and D21 after hybridization with the liquid mixtures 2, 3 and 4 were measured.

From the result of the measurement of current values, it seemed that both of the methods 1 and 2 exhibited the similar tendency as follows.

That is, for the liquid mixture 1, the hybridization reaction occurred only with the reaction electrode D11; in the case of liquid mixture 2, the hybridization reaction occurred only with reaction electrode D12; in the case of liquid mixture 3, the hybridization reaction occurred with the reaction electrodes D11 and D12; and in the case of liquid mixture 4, no hybridization reaction was observed with any of the reaction electrodes D11-D21.

Therefore, a target DNA in each liquid mixture was reliably determined in the example.

### INDUSTRIAL APPLICABILITY

As described above, according to the charged substance detector of the present invention, a plurality of reaction electrodes are arranged in matrix on the detection panel and made selectively energizable to each reaction electrodes through the matrix of wires. Therefore, it is possible to selectively energize a reaction electrode at a desired position without individual connection wires to the reaction electrodes and to make a reaction charged substance specifically react with a target charged substance on the selected reaction electrode. This allows a charged substance to be detected easily and quickly without complicating the construction of the detector.

1. A charged substance detector comprising:
a detection panel having a plurality of reaction electrodes arrayed in matrix, on which are immobilized reaction charged substances reacting specifically with target charged substances; and
a conduction control device which has a matrix of wires crossing each other at points corresponding to the matrix-arrayed reaction electrodes of the detection panel, and which can selectively energize the reaction electrodes through the matrix of wires.

2. The charged substance detector according to claim 1, wherein the target charged substance is DNA or gene.

3. The charged substance detector according to claim 1, wherein the detection panel comprises a first board having the plurality of reaction electrodes arrayed in matrix on which the reaction charged substances that specifically react with the target charged substances, and the second board opposing the first board and having an electrode to apply a voltage between it and the selected reaction electrode in the matrix.

4. The charged substance detector according to claim 3, wherein the second board has a plurality of electrodes arranged at positions corresponding to the matrix-arrayed reaction electrodes of the first board.

5. The charged substance detector according to claim 1, wherein the reaction electrodes, on which are immobilized the reaction charged substances that specifically react with the target charged substances, has an immobilizing layer for the reaction charged substances.

6. The charged substance detector according to claim 1, wherein the conduction control device has switch elements connected between the plurality of reaction electrodes arrayed in matrix and the matrix of wires and selectively turns on or off the switch elements to apply a predetermined voltage to a specific reaction electrode.

7. The charged substance detector according to claim 1, wherein the conduction control device has a conduction state detector for detecting a conduction state of each reaction electrode.

8. A method of using the charged substance detector according to claim 1, comprising the step of:
applying a voltage to a specific reaction electrode of the detection panel to immobilize a predetermined reaction charged substance to the specified reaction electrode.

9. A method of using the charged substance detector according to claim 1, comprising the steps of:
to determine whether the same target charged substance is present in a plurality of samples, immobilizing a predetermined reaction charged substance on a required number of reaction electrodes of the detection panel; and
successively applying a predetermined voltage to specific reaction electrodes corresponding to each of the different samples to successively allow the predetermined reaction charged substance to react specifically with the target charged substance in each sample.

10. A method of using the charged substance detector according to claim 1, comprising the steps of:
to determine whether a plurality of target charged substances are present in one sample, successively immobilizing different reaction charged substances to each of specific reaction electrodes of the detection panel; and
applying a predetermined voltage to each of the reaction electrodes to successively allow the different reaction charged substances to react specifically with the corresponding target charged substances in the sample.

11. A method of using the charged substance detector according to claim 1, comprising the steps of:
to determine whether a plurality of target charged substances are present in a plurality of samples, successively immobilizing different reaction charged substances to each of reaction electrodes of the detection panel; and
applying a predetermined voltage to each of the reaction electrodes corresponding to each of the samples to successively allow the reaction charged substances on the reaction electrodes to react specifically with the corresponding target charged substances in the samples.

12. A detection panel used in the charged substance detector according to claim 1, wherein the reaction charged substances the number of which is equal to that of inspection items are assigned and immobilized to predetermined addresses of the reaction electrodes.


SEQUENCE LISTING (5 sequences)

SEQ ID NO: 1 - DNA, 21 bp, Artificial Sequence
  Description of Artificial Sequence Synthetic DNA
gacggaacagctttgaggtgc

SEQ ID NO: 2 - DNA, 18 bp, Artificial Sequence
  Description of Artificial Sequence Synthetic DNA
tgacggaggttgtgaggc

SEQ ID NO: 3 - DNA, 21 bp, Artificial Sequence
  Description of Artificial Sequence Synthetic DNA
gcacctcaaagctgttccgtc

SEQ ID NO: 4 - DNA, 18 bp, Artificial Sequence
  Description of Artificial Sequence Synthetic DNA
gcctcacaacctccgtca

SEQ ID NO: 5 - DNA, 21 bp, Artificial Sequence
  Description of Artificial Sequence Synthetic DNA
gcacagaggaagagaatctcc